(19) United States
(12) Patent Application Publication
(10) Pub. No.: US 2019/0074091 A1
(43) Pub. Date: Mar. 7, 2019

(54) INFORMATION PROCESSING DEVICE, METHOD OF PROCESSING INFORMATION, AND PROGRAM

(71) Applicant: SONY CORPORATION, TOKYO (JP)

(72) Inventors: KAZUKI AISAKA, KANAGAWA (JP); KENJI YAMANE, KANAGAWA (JP)

(21) Appl. No.: 16/083,888

(22) PCT Filed: Feb. 21, 2017
(86) PCT No.: PCT/JP2017/006433

§ 371 Date: Sep. 10, 2018

(30) Foreign Application Priority Data
May 17, 2016 (JP) 2016-098740

(51) Int. Cl. G16H 50/30; G06F 19/24; G16H 15/00

(52) U.S. Cl. CPC G16H 50/30; G16H 15/00; G06F 19/24

(57) ABSTRACT

[Object] To provide a technology capable of generating data more suitable for the subject of the genetic test. [Solution] There is provided an information processing device including: an acquisition unit that acquires first input information that includes at least a first genetic test result and a first health examination result; and a computing unit that acquires a weight of each of a plurality of data on a basis of a category to which the first input information belongs and specifies the data to be notified from the plurality of data on a basis of the weight of each of the plurality of data.

### TECHNICAL FIELD

The present disclosure relates to an information processing device, a method of processing information, and a program.

### BACKGROUND ART

In recent years, with the advent of a next generation sequencer, it becomes possible to perform a genetic test inexpensively and quickly using the next generation sequencer. A genetic test result is used in various situations. For example, a technology for recommending an improvement of life style, a dieting method, and the like to a subject on the basis of the genetic test result is disclosed (e.g., see Patent Literature 1).

### CITATION LIST

### Patent Literature

Patent Literature 1: JP 2015-64884A

### DISCLOSURE OF INVENTION

### Technical Problem

However, it is desirable to provide a technology capable of generating data more suitable for the subject of the genetic test.

### Solution to Problem

According to the present disclosure, there is provided an information processing device including: an acquisition unit that acquires first input information that includes at least a first genetic test result and a first health examination result; and a computing unit that acquires a weight of each of a plurality of data on a basis of a category to which the first input information belongs and specifies the data to be notified from the plurality of data on a basis of the weight of each of the plurality of data.

According to the present disclosure, there is provided an information processing method including: acquiring first input information that includes at least a first genetic test result and a first health examination result; and acquiring a weight of each of a plurality of data on a basis of a category to which the first input information belongs and specifying the data to be notified from the plurality of data on a basis of the weight of each of the plurality of data by a processor.

According to the present disclosure, there is provided a program that causes a computer to function as an information processing device that includes an acquisition unit that acquires first input information that includes at least a first genetic test result and a first health examination result, and a computing unit that acquires a weight of each of a plurality of data on a basis of a category to which the first input information belongs and specifies the data to be notified from the plurality of data on a basis of the weight of each of the plurality of data.

### Advantageous Effects of Invention

As described above, according to the present disclosure, there can be provided the technology capable of generating the data more suitable for the subject of the genetic test. Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a diagram illustrating a configuration example of an information processing system according to an embodiment of the present disclosure.

FIG. 2 is a block diagram illustrating a functional configuration example of a terminal according to the present embodiment.

FIG. 3 is a block diagram illustrating a functional configuration example of an information processing device according to the present embodiment.

FIG. 4 is a diagram describing how a database is constructed.

FIG. 5 is a diagram illustrating an example of a category to which an item related to a disease A belongs and a weight of a comment for the disease A.

FIG. 6 is a diagram illustrating an example of a category to which an item related to a disease B belongs and a weight of a comment for the disease B.

FIG. 7 is a diagram illustrating an association example of the category and the weight of each of a plurality of comments.

FIG. 8 is a diagram describing how the comment is generated.

FIG. 9 is a diagram describing feedback of information indicating whether or not an improvement is made by notification of the comment to be notified.

FIG. 10 is a diagram describing a calculation example of an improvement probability by the comment to be notified.

FIG. 11 is a diagram illustrating an example of the plurality of comments.

FIG. 12 is a flow chart illustrating an operation example of the database construction.

FIG. 13 is a flow chart illustrating a first operation example of an update of a comment improvement probability.

FIG. 14 is a flow chart illustrating a first operation example of an update of a comment improvement probability.

FIG. 15 is a flow chart illustrating an operation example of a preparation of an analysis report describing the comment to be notified.

FIG. 16 is a block diagram illustrating a hardware configuration example of a terminal.

FIG. 17 is a block diagram illustrating a hardware configuration example of an information processing device.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that, in this description and the drawings, structural elements that have substantially the same function and structure are sometimes distinguished from each other using different numerals after the same reference sign. However, when there is no need in particular to distinguish structural elements that have substantially the same function and structure, the same reference sign alone is attached.

### INDUSTRIAL APPLICABILITY

Moreover, the description will be given in the following order.

### 0. Background

1. Embodiment of present disclosure
1.1. System configuration example
1.2. Functional configuration example
1.3. Details of functions of information processing system
1.4. Operation example of information processing system
1.5. Hardware configuration example

### 2. Conclusion

### 0. BACKGROUND

In recent years, with the advent of a next generation sequencer, it becomes possible to perform a genetic test inexpensively and quickly using the next generation sequencer. A genetic test result is used in various situations. For example, a technology for recommending an improvement of life style, a dieting method, and the like to a subject on the basis of the genetic test result is disclosed (e.g., see JP 2015-64884A).

However, it is difficult to generate the data sufficiently suitable for the subject of the genetic test simply by generating the data to be provided to the subject on the basis of the genetic test result. Thus, in this specification, the description will mainly focus on a technology capable of generating the data more suitable for the subject of the genetic test. Further, according to the present technology, it becomes possible to prevent a variation of the data caused by the difference in creators who generate the data and reduce a burden of generating the data by the creator.

Note that, in the present embodiment, the description will mainly focus on a case where the creator is a medical doctor and the data generated by the creator is a comment provided from the medical doctor to the subject. However, the creator is not limited to the medical doctor. Further, the data generated by the creator is not limited to the comment provided from the medical doctor to the subject. For example, the data generated by the creator may be information regarding a medicine prescribed to the subject, and the like. Note that the comment provided from the medical doctor to the subject may include an instruction provided from the medical doctor to the subject.

### 1. EMBODIMENT OF PRESENT DISCLOSURE

### 1.1. System Configuration Example

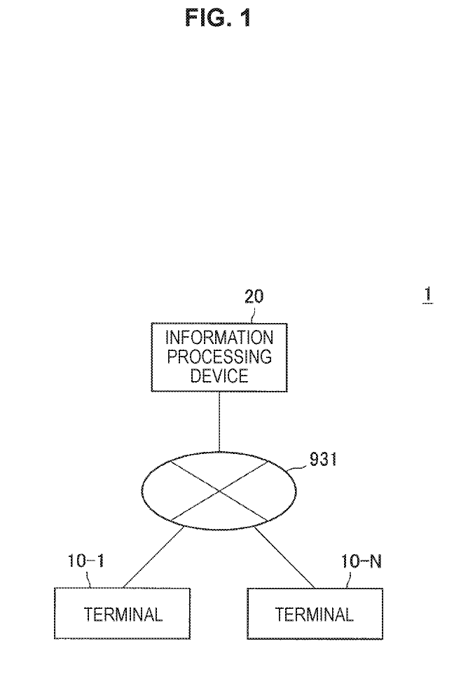

A configuration example of an information processing system according to an embodiment of the present disclosure is now described with reference to the drawings. FIG. 1 is a diagram illustrating a configuration example of the information processing system according to an embodiment of the present disclosure. As illustrated in FIG. 1, the information processing system 1 according to the present embodiment includes terminals 10-1 to 10-N (N is a natural number) and an information processing device 20. The terminals 10-1 to 10-N and an information processing device 20 are capable of communicating with each other via a communication network 931.

Moreover, in the example illustrated in FIG. 1, the form of the terminals 10-1 to 10-N is not limited to a particular form. In one example, the terminals 10-1 to 10-N may be a game console, a smartphone, a mobile phone, a tablet terminal, and a personal computer (PC). In addition, the information processing device 20 is assumed to be a computer such as a server.

The configuration example of the information processing system 1 according to the present embodiment is described above.

### 1.2. Functional Configuration Example

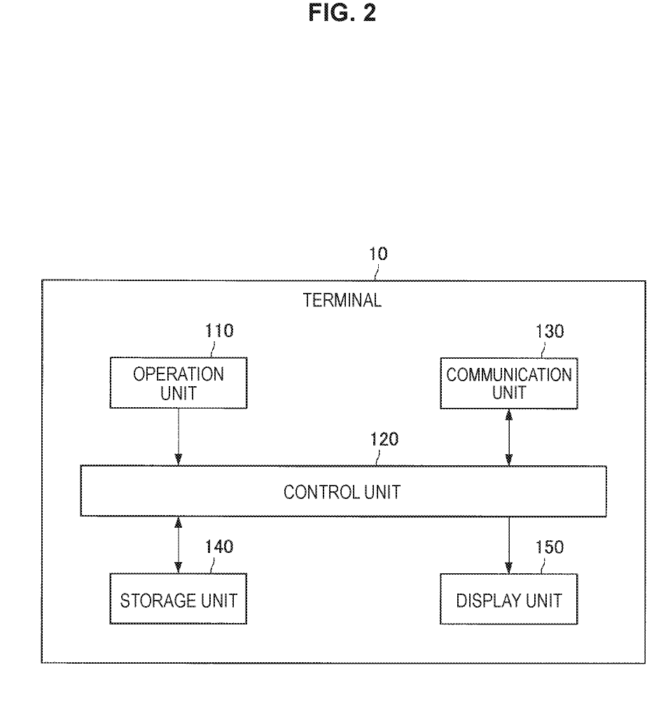

Subsequently, a functional configuration example of the terminal 10 according to the present embodiment is described. FIG. 2 is a block diagram illustrating a functional configuration example of the terminal 10 according to the present embodiment. As illustrated in FIG. 2, the terminal 10 includes an operation unit 110, a control unit 120, a communication unit 130, a storage unit 140, and a display unit 150. These functional blocks included in the terminal 10 are described below.

The operation unit 110 has a function of receiving a subject's operation. In one example, the operation unit 110 may include an input device such as a mouse and a keyboard. In addition, the operation unit 110 may include a touch panel as long as it has a function of receiving the subject's operation. A type of the touch panel to be employed is not limited to a particular type, and may be an electrostatic capacitive, resistive-film, infrared, or ultrasonic type. In addition, the operation unit 110 may include a camera.

The control unit 120 controls the respective units included in the terminal 10. Moreover, the control unit 120 may include, in one example, a central processing unit (CPU), or the like. In the case where the control unit 120 includes a processing device such as a CPU, such a processing device may include an electronic circuit.

The communication unit 130 has a function of communicating with the information processing device 20. In one example, the communication unit 130 includes a communication interface. In one example, the communication unit 130 is capable of communicating with the information processing device 20 via the communication network 931 (FIG. 1).

The storage unit 140 is a recording medium that stores a program to be executed by the control unit 120 and stores data necessary for execution of the program. In addition, the storage unit 140 temporarily stores data for computation by the control unit 120. The storage unit 140 may be a magnetic storage unit device, a semiconductor storage device, an optical storage device, or a magneto-optical storage device.

The display unit 150 has a function of displaying various types of information. In one example, the display unit 150 may be a liquid crystal display, an organic electro-luminescence (EL) display, or a head-mounted display (HMD). However, the display unit 150 may be other forms of display as long as it has the function of displaying various types of information.

The functional configuration example of the terminal 10 according to the present embodiment is described above.

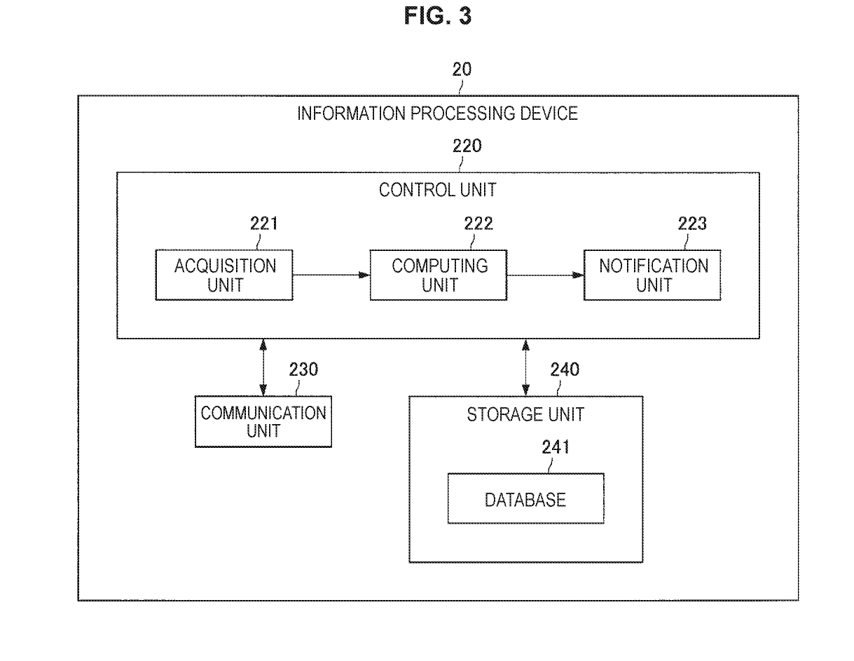

Subsequently, the functional configuration example of the information processing device 20 according to the present embodiment is described. FIG. 3 is a block diagram illustrating the functional configuration example of the information processing device 20 according to the present embodiment. As illustrated in FIG. 3, the information processing device 20 includes a control unit 220, a communication unit 230, and a storage unit 240. These functional blocks included in the information processing device 20 are described below.

The control unit 220 controls the respective units included in the information processing device 20. As illustrated in FIG. 3, the control unit 220 includes an acquisition unit 221, a computing unit 222 and a notification unit 223. Details of these functional blocks equipped in the control unit 220 will be described later. Moreover, the control unit 220 may include, in one example, a central processing unit (CPU), or the like. In the case where the control unit 220 includes a processing device such as a CPU, such a processing device may include an electronic circuit.

The communication unit 230 has a function of communicating with the terminals 10-1 to 10-N. In one example, the communication unit 230 includes a communication interface. In one example, the communication unit 230 is capable of communicating with the terminals 10-1 to 10-N via the communication network 931 (FIG. 1).

The storage unit 240 is a recording medium that stores a program to be executed by the control unit 220 and stores data necessary for execution of the program. In this specification, an example where the storage unit 240 stores a database 241 as shown in FIG. 3 will be described. In addition, the storage unit 240 temporarily stores data for computation by the control unit 220. The storage unit 240 may be a magnetic storage unit device, a semiconductor storage device, an optical storage device, or a magneto-optical storage device.

The functional configuration example of the information processing device 20 according to the present embodiment is described above.

### 1.3. Details of Functions of Information Processing System

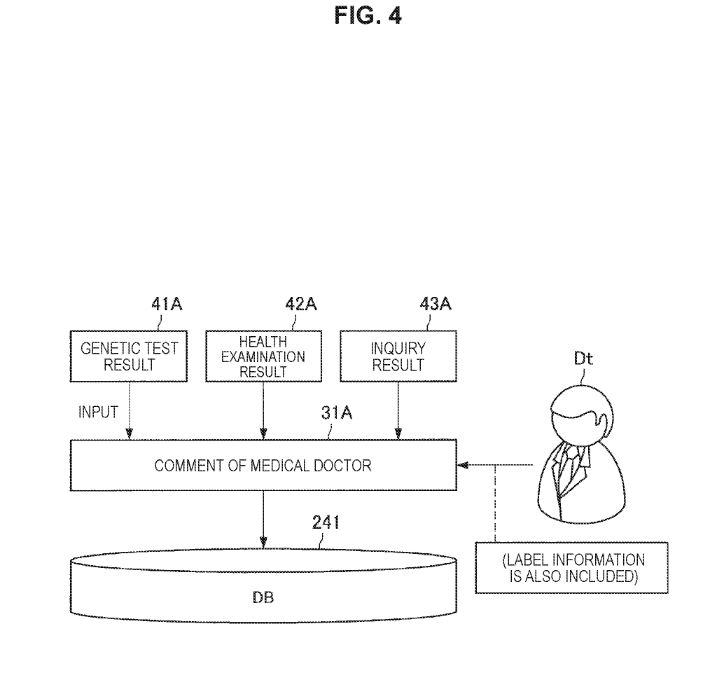

Subsequently, details of functions of the information processing system 1 will be described. FIG. 4 is a diagram describing how a database is constructed. As shown in FIG. 4, when a medical doctor Dt acquires a genetic test result 41A, a health examination result 42A, and an inquiry result 43A, the medical doctor Dt generates a comment 31A using the terminal 10 while viewing these pieces of information. The genetic test result 41A, the health examination result 42A, the inquiry result 43A, and the comment 31A are sent from the terminal 10 to the information processing device 20 and stored in the database 241 in the information processing device 20.

The database 241 in the information processing device 20 stores the genetic test result 41A, the health examination result 42A, and the inquiry result 43A as input information (hereinafter also referred to as “input information A”). Further, the database 241 in the information processing device 20 stores the comment 31A associated with the input information A. Note that the input information A is only required to include at least the genetic test result 41A and the health examination result 42A and may further include the inquiry result 43A.

In this process, the medical doctor Dt does not always generate the comment for all items in the genetic test result 41A, the health examination result 42A, and the inquiry result 43A, and there may be a case where the medical doctor Dt generates the comment for a part of items in the genetic test result 41A, the health examination result 42A, and the inquiry result 43A. Thus, the medical doctor Dt may be allowed to give a label to the item associated with the comment. In such a case, the item to which the label is given is sent from the terminal 10 to the information processing device 20 and stored in the database 241 in the information processing device 20.

In this manner, a plurality of combinations of the input information A and the comment associated with the input information A are stored in the database 241. The computing unit 222 performs learning processing on a category to which the input information A stored in the database 241 belongs and data associated with the input information A and thereby calculates a weight of each of a plurality of comments in each category. The learning processing performed by the computing unit 222 is not particularly limited. For example, Bayesian inference or other machine learning methods may be used for the learning processing.

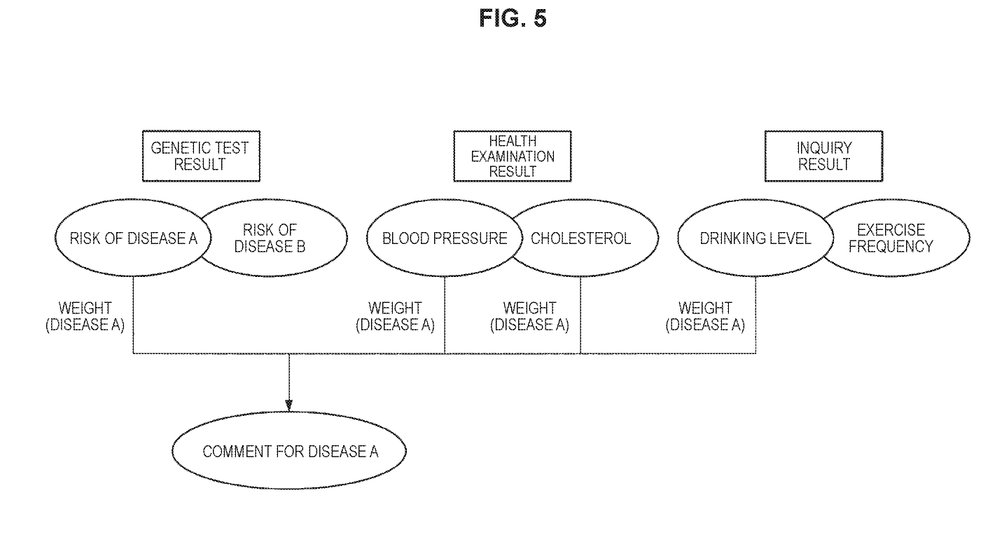

FIG. 5 is a diagram illustrating an example of the category to which the item related to a disease A belongs and the weight of the comment for the disease A. As shown in FIG. 5, the computing unit 222 calculates, through the learning processing, the category to which the item related to the disease A belongs (the item “risk of disease A” in the genetic test result, the item “blood pressure” in the health examination result, the item “cholesterol” in the health examination result, and the item “drinking level” in the inquiry result) and the weight of the comment for the disease A. For example, the item related to the disease A may be specified in advance by an expert such as a medical doctor or automatically learned.

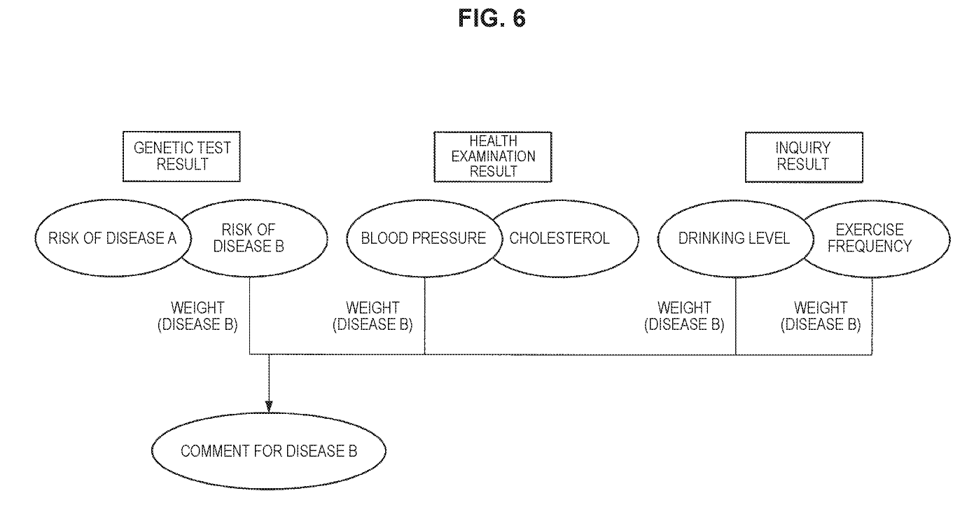

FIG. 6 is a diagram illustrating an example of the category to which the item related to a disease B belongs and the weight of the comment for the disease B. As shown in FIG. 6, the computing unit 222 calculates, through the learning processing, the category to which the item related to the disease B belongs (the item “risk of disease B” in the genetic test result, the item “blood pressure” in the health examination result, the item “drinking level” in the inquiry result, and the item “exercise frequency” in the inquiry result) and the weight of the comment for the disease B. For example, the item related to the disease B may be specified in advance by an expert such as a medical doctor or automatically learned.

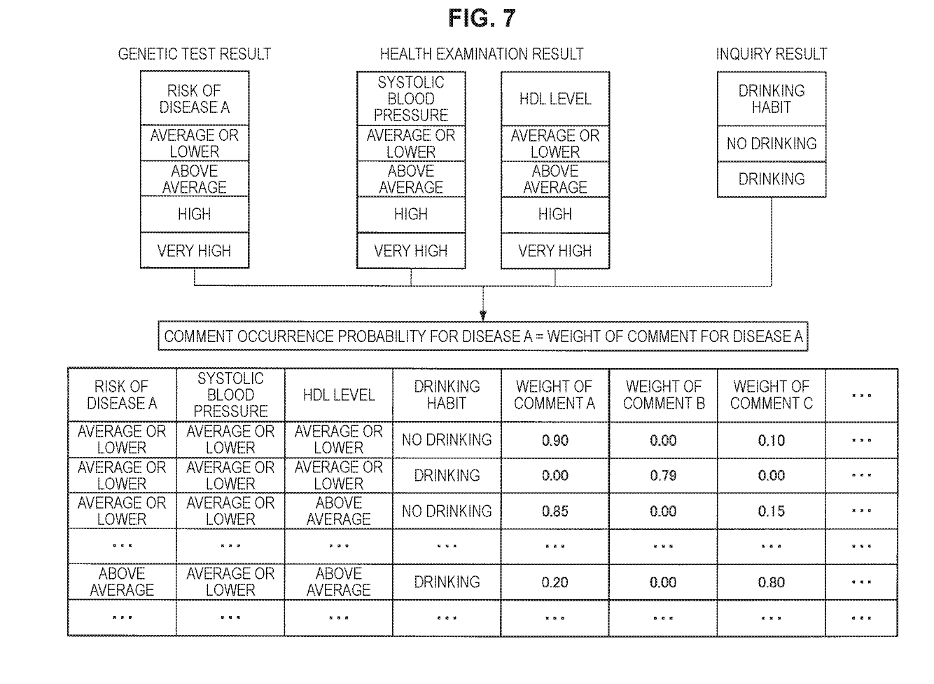

More specifically, the computing unit 222 may specify the weight of the specific comment on the basis of a ratio of an occurrence frequency of the specific comment with respect to the occurrence frequency of the plurality of comments in the database 241 (hereinafter, also simply referred to as a “comment occurrence probability”). FIG. 7 is a diagram illustrating an association example of the category and the weight of each of the plurality of comments. As shown in FIG. 7, for example, the computing unit 222 may use the comment occurrence probability for the disease A as the weight of the comment for the disease A.

For example, in the example shown in FIG. 7, the weight of a comment A for the category on the uppermost stage (risk of disease A “average or lower”, systolic blood pressure “average or lower”, HDL level “average or lower”, drinking habit “no drinking”) is specified as “0.90”. Similarly, the weight of a comment B for the category on the uppermost stage is specified as “0.00”, and the weight of a comment C is specified as “0.10”.

Note that, in the example shown in FIG. 7, the category is defined in accordance with levels (average or lower, above average, high, very high) of “risk of disease A” in the genetic test result. Further, the category is defined in accordance with levels (average or lower, above average, high, very high) of “systolic blood pressure” and “HDL level (HDL cholesterol level)” in the health examination result. As these levels, for example, a criteria category described in a reference (Japan Society of Ningen Dock, a new criteria category PDF (revised in April 2016) http://www.ningen-dock.jp/other/inspection) may be used. In this case, the systolic blood pressure of 160 mmHg or higher is considered as a very high level. Further, the category is defined in accordance with levels (no drinking, drinking) of “drinking habit” in the inquiry item.

Note that, in this specification, the description mainly focused on the example where the computing unit 222 generates the comment occurrence probability using the ratio of the occurrence frequency of the specific comment with respect to the occurrence frequency of the plurality of comments in the database 241. However, the comment occurrence probability may be generated by other methods. For example, the comment occurrence probability may be generated on the basis of statistics information for epidemiology. Further, in this specification, the example of using “systolic blood pressure” and “HDL level” as the item in the health examination result has been described, however, “diastolic blood pressure”, “LDL level”, and the like may be used.

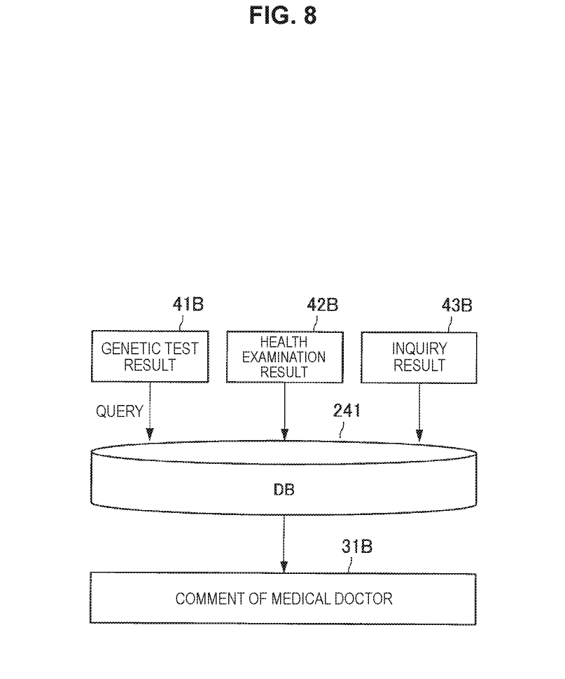

FIG. 8 is a diagram describing how the comment is generated. As shown in FIG. 8, when the medical doctor Dt acquires a genetic test result 41B, a health examination result 42B, and an inquiry result 43B, the genetic test result 41B, the health examination result 42B, and the inquiry result 43B, which constitute input information (hereinafter also referred to as “input information B”), are sent from the terminal 10 to the information processing device 20 as a query. Note that the input information B is only required to include at least the genetic test result 41B and the health examination result 42B and may further include the inquiry result 43B.

In this process, the medical doctor Dt does not always wish to generate the comment for all items in the genetic test result 41B, the health examination result 42B, and the inquiry result 43B, and there may be a case where the medical doctor Dt wishes to generate the comment for a part of items in the genetic test result 41B, the health examination result 42B, and the inquiry result 43B. Thus, the medical doctor Dt may be allowed to give a label to the item associated with the comment. In such a case, the item to which the label is given is sent from the terminal 10 to the information processing device 20 as the query.

The acquisition unit 221 in the information processing device 20 acquires the input information B. The computing unit 222 then acquires the weight of each of the plurality of comments on the basis of the category to which the input information B belongs and specifies a comment 31B to be notified from the plurality of comments on the basis of the weight of each of the plurality of comments. Having such a configuration makes it possible to generate the comment more suitable for the subject of the genetic test. Further, having such a configuration makes it possible to prevent a variation of the comment caused by the difference in the medical doctors who generate the comment and reduce a burden of generating the comment by the medical doctor.

In this process, no particular limitation is imposed on how the comment 31B to be notified is specified. As an example, the computing unit 222 may specify the comment having the largest weight among the weights of the plurality of comments as the comment 31B to be notified. The comment 31B to be notified, which has been specified by the computing unit 222, is notified to the terminal 10 by the notification unit 223. The terminal 10 receives the comment 31B to be notified by the communication unit 130 and displays the comment 31B to be notified by the display unit 150.

In the terminal 10, an analysis report describing the comment 31B to be notified may be created. In this process, the analysis report may be added with a comment by the medical doctor in addition to the comment 31B to be notified. In the case where a new comment is added by the medical doctor to the analysis report describing the comment 31B to be notified, the computing unit 222 may add the new comment to the existing plurality of comments.

Further, when the comment 31B to be notified is notified to the terminal 10, the occurrence frequency of the comment 31B to be notified increases, thereby increasing the occurrence probability of the comment 31B to be notified. On the other hand, when the comment 31B to be notified is notified to the terminal 10, the occurrence probabilities of the comments other than the comment 31B to be notified are supposedly reduced. Thus, the computing unit 222 preferably updates the occurrence probability of each of the plurality of comments when the comment 31B to be notified is notified to the terminal 10.

Further, when the comment 31B to be notified is notified to the terminal 10, it is preferable to provide feedback as to whether or not an improvement is made by notification of the comment 31B to be notified. Specifically, the computing unit 222 preferably reflects an improvement probability by the comment 31B to be notified on the weight of the comment 31B to be notified as feedback to the notification of the comment 31B to be notified and thereby updates the weight of the comment 31B to be notified.

There is no limitation on how to calculate the improvement probability by the comment 31B to be notified. As an example, the computing unit 222 may calculate the improvement probability by the comment 31B to be notified on the basis of information indicating whether or not an improvement is made by the notification of the comment 31B to be notified. For example, the computing unit 222 may calculate the improvement probability by the comment 31B to be notified using a ratio of a frequency in which an improvement is made by the comment 31B to be notified with respect to the total frequency in which the comment 31B to be notified is notified.

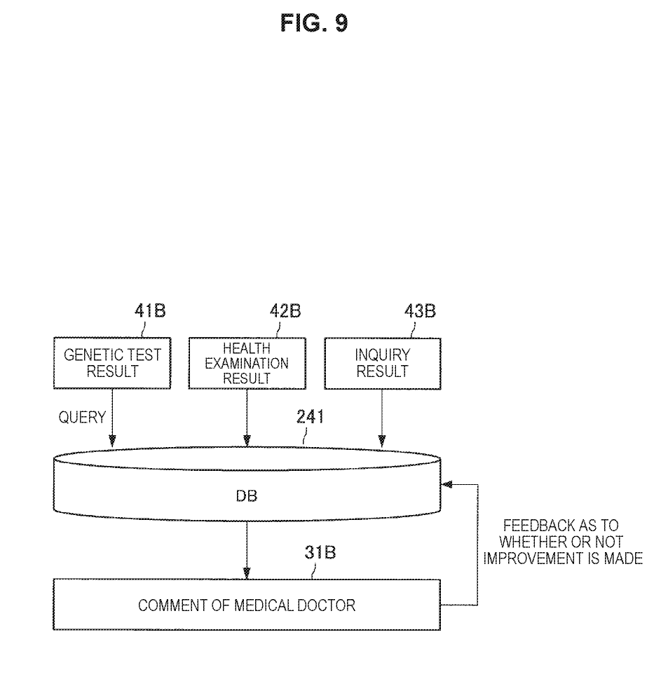

FIG. 9 is a diagram describing the feedback of the information indicating whether or not an improvement is made by the notification of the comment 31B to be notified. As shown in FIG. 9, the information indicating whether or not an improvement is made by the notification of the comment 31B to be notified is preferably fed back.

There is no limitation on how to obtain the information indicating whether or not an improvement is made by the notification of the comment 31B to be notified. As an example, the information indicating whether or not an improvement is made may be manually inputted into the terminal 10 by the medical doctor. Then, the information indicating whether or not an improvement is made, inputted in this manner, may be acquired by the acquisition unit 221.

Alternatively, the information indicating whether or not an improvement is made may be automatically calculated by the computing unit 222. More specifically, the computing unit 222 may calculate the information indicating whether or not an improvement is made on the basis of the health examination result in the past and the health examination result at present. The health examination result at present may be the same as the health examination result 42B described above. For example, the computing unit 222 may calculate the information indicating whether or not an improvement is made on the basis of the categories to which the health examination result in the past and the health examination result at present belong.

For example, the computing unit 222 may determine whether or not an improvement is made on the basis of whether or not the category to which the health examination result at present belongs is better than the category to which the health examination result in the past belongs. For example, the computing unit 222 can determine that an improvement is made in a case where the item classified into the category “high” in the health examination result in the past is now classified into the better category such as “above average” or “average or lower” in the health examination result at present.

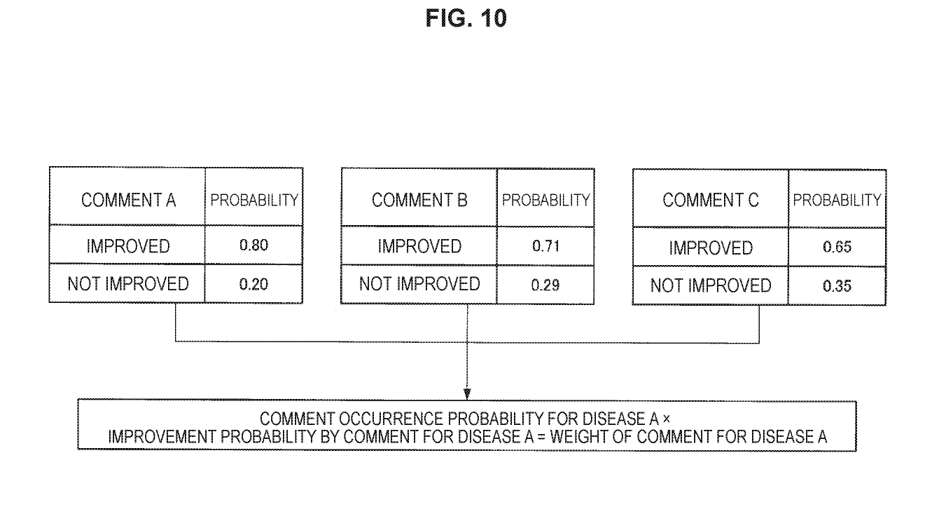

Note that there is no limitation as to how the computing unit 222 reflects the improvement probability by the comment to be notified on the weight of the comment to be notified. FIG. 10 is a diagram describing a calculation example of the improvement probability by the comment to be notified. A case where the comment to be notified is the comment for the disease A is described as an example. As shown in FIG. 10, the computing unit 222 may calculate the weight of the comment for the disease A by multiplying the comment occurrence probability for the disease A by the improvement probability by the comment for the disease A.

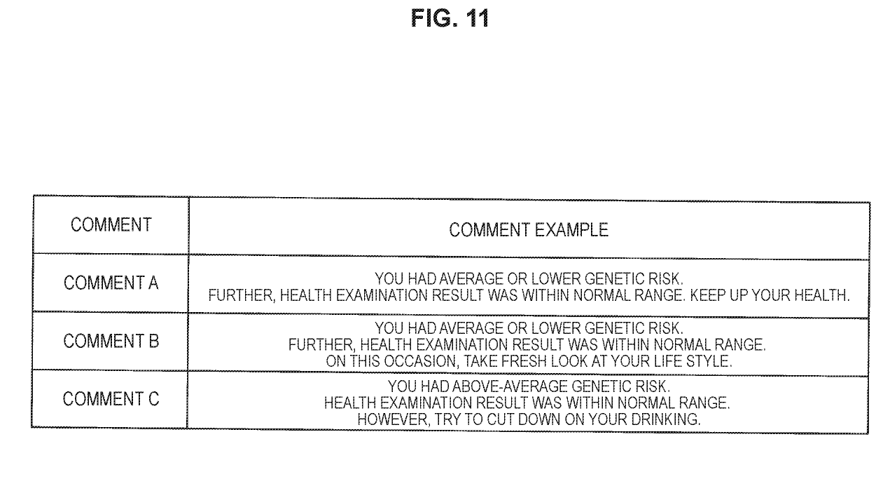

FIG. 11 is a diagram illustrating an example of the plurality of comments. Referring to FIG. 11, a comment A, comment B, and comment C are listed as an example of the plurality of comments. However, the comment is not limited to these three kinds, and it is only required that there are a plurality of kinds of comments. Further, contents of each of the plurality of comments is not particularly limited. Note that, in this example, the comment example for the disease A is shown, however, other comments such as the comment for the disease B can be expressed in a similar manner. Further, the comment for a certain disease (e.g., the comment causing a high improvement rate) may be made usable as the comment for other diseases.

Further, although the example of using a simple probability model is described above, a probability model such as a Bayesian network may be used. The Bayesian network is described, for example, in a reference (Introduction to Bayesian Network (1), Suyari Hiroki, MEDICAL IMAGING TECHNOLOGY Vol. 21 No. 4 Sep. 2003, www.ne.jp/asahi/hiroki/suyari/BayesianNetworkIntro1.pdf).

The details of functions of the information processing system 1 are described above.

### 1.4. Operation Example of Information Processing System

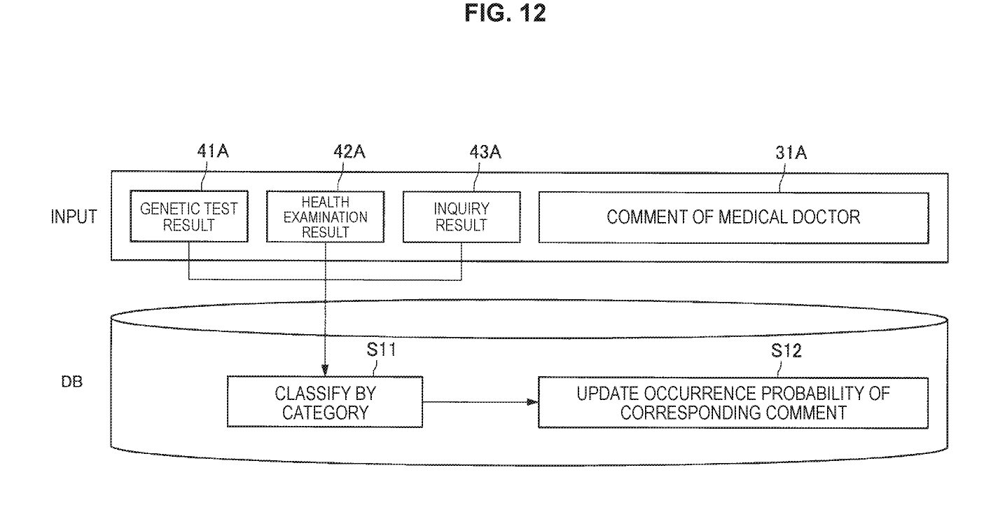

Subsequently, an operation example of the information processing system 1 is described. FIG. 12 is a flow chart illustrating an operation example of the database construction. As shown in FIG. 12, when the medical doctor acquires the genetic test result 41A, the health examination result 42A, and the inquiry result 43A, the medical doctor generates the comment 31A using the terminal 10 while viewing these pieces of information. The genetic test result 41A, the health examination result 42A, the inquiry result 43A, and the comment 31A are sent from the terminal 10 to the information processing device 20.

The computing unit 222 in the information processing device 20 classifies a combination of the genetic test result 41A, the health examination result 42A, and the inquiry result 43A by category and determines the category to which the combination belongs (S11). Further, the computing unit 222 performs the learning processing using the category thus determined and the comment 31A to calculate the occurrence probability of the comment associated with the category and then updates the occurrence probability of such a comment (S12).

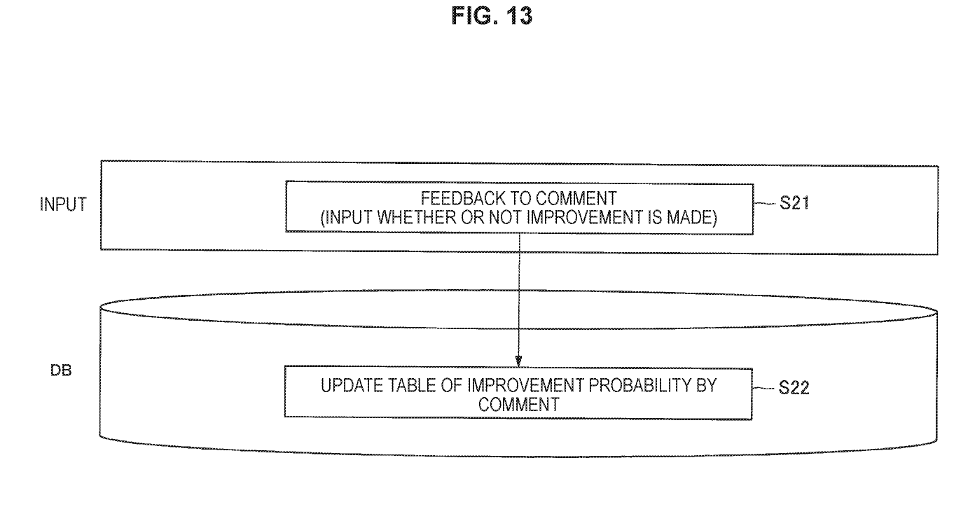

FIG. 13 is a flow chart illustrating a first operation example of the update of the comment improvement probability. As shown in FIG. 13, when information indicating whether or not an improvement is made is inputted, the information indicating whether or not an improvement is made is acquired by the acquisition unit 221 (S21). The computing unit 222 calculates the improvement probability by the comment to be notified on the basis of the information indicating whether or not an improvement is made by the notification of the comment to be notified and then updates a table of the improvement probability by the comment on the basis of the improvement probability by the comment to be notified (S22).

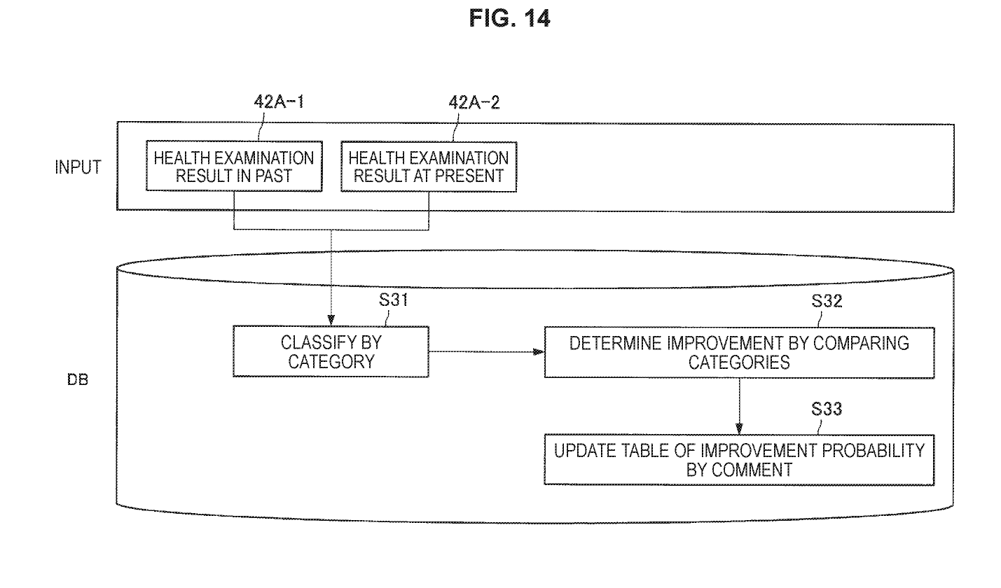

FIG. 14 is a flow chart illustrating a second operation example of the update of the comment improvement probability. As shown in FIG. 14, when a health examination result 42A-1 in the past and a health examination result 42A-2 at present are inputted, the computing unit 222 classifies each of the health examination result 42A-1 in the past and the health examination result 42A-2 at present by category and determines the category to which each result belongs (S31). The computing unit 222 calculates the information indicating whether or not an improvement is made by comparing the categories to which the health examination result in the past and the health examination result at present belong (S32).

Subsequently, the computing unit 222 calculates the improvement probability by the comment to be notified on the basis of the information indicating whether or not an improvement is made by the notification of the comment to be notified and then updates the table of the improvement probability by the comment on the basis of the improvement probability by the comment to be notified (S33).

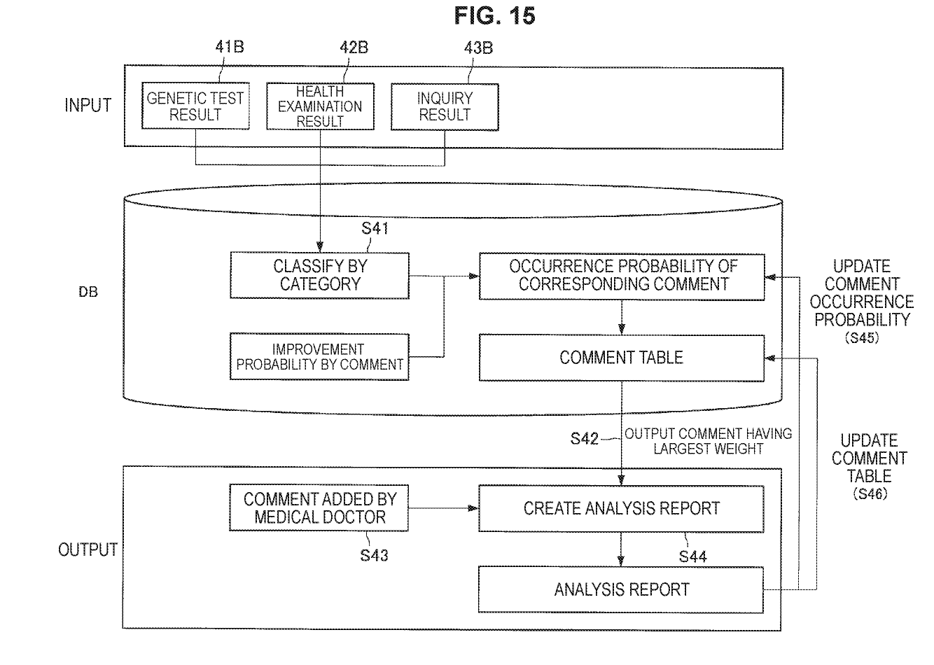

FIG. 15 is a flow chart illustrating an operation example of a preparation of an analysis report describing the comment to be notified. As shown in FIG. 15, the genetic test result 41B, the health examination result 42B, and the inquiry result 43B, which constitute the input information B, are sent from the terminal 10 to the information processing device 20 as a query. The acquisition unit 221 in the information processing device 20 acquires the input information B. The computing unit 222 then classifies the input information B by category and determines the category to which the input information B belongs (41).

Then, the computing unit 222 acquires the improvement probability by the comment and the comment occurrence probability in each of the plurality of comments associated with the category to which the input information B belongs. The computing unit 222 calculates the weight of each of the plurality of comments associated with the category to which the input information B belongs by multiplying the improvement probability by the comment by the comment occurrence probability. The computing unit 222 acquires the comment having the largest weight from a comment table among the weights of the plurality of comments. The notification unit 223 outputs such a comment as the comment to be notified (S42).

When the comment to be notified is received by the terminal 10, the analysis report describing the comment to be notified is created. (S44). In this process, the analysis report may be added with a comment by the medical doctor in addition to the comment 31B to be notified (S43). When a new comment is added to the analysis report by the medical doctor, the computing unit 222 updates the comment occurrence probability (S45) and also updates the comment table by adding the new comment to the comment table (S46).

The operation example of the information processing system 1 is described above.

### 1.5. Hardware Configuration Example

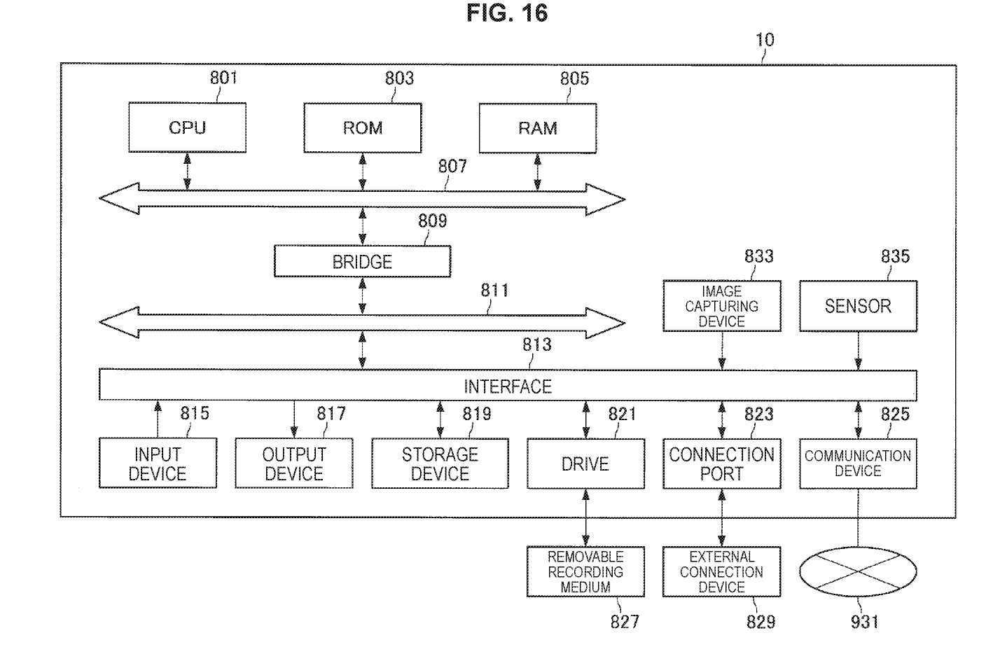

Next, the hardware configuration of the terminal 10 according to an embodiment of the present disclosure is described with reference to FIG. 16. FIG. 16 is a block diagram illustrating a hardware configuration example of the terminal 10 according to an embodiment of the present disclosure.

As illustrated in FIG. 16, the terminal 10 includes a central processing unit (CPU) 801, a read-only memory (ROM) 803, and a random-access memory (RAM) 805. In addition, the terminal 10 may include a host bus 807, a bridge 809, an external bus 811, an interface 813, an input device 815, an output device 817, a storage device 819, a drive 821, a connection port 823, and a communication device 825. The terminal 10 may further include an image capturing device 833 and a sensor 835 as necessary. In conjunction with, or in place of, the CPU 801, the terminal 10 may have a processing circuit called a digital signal processor (DSP) or application specific integrated circuit (ASIC).

The CPU 801 functions as an arithmetic processing unit and a control unit, and controls the whole operation in the terminal 10 or a part thereof in accordance with various programs recorded in the ROM 803, the RAM 805, the storage device 819, or a removable recording medium 827. The ROM 803 stores programs, operation parameters, or the like used by the CPU 801. The RAM 805 temporarily stores programs used in the execution by the CPU 801, parameters that vary as appropriate in the execution, or the like. The CPU 801, the ROM 803, and the RAM 805 are connected with each other via the host bus 807 that includes an internal bus such as a CPU bus. Furthermore, the host bus 807 is connected to the external bus 811 such as peripheral component interconnect/interface (PCI) bus via the bridge 809.

The input device 815 is, in one example, a device operated by a user, such as a mouse, a keyboard, a touch panel, a button, a switch, and a lever. The input device 815 may include a microphone for detecting user's speech. The input device 815 may be, in one example, a remote control device using infrared rays or other radio waves, or may be an external connection device 829 such as a cellular phone that supports the operation of the terminal 10. The input device 815 includes an input control circuit that generates an input signal on the basis of the information input by the user and outputs it to the CPU 801. The user operates the input device 815 to input various data to the terminal 10 and to instruct the terminal 10 to perform a processing operation. In addition, the image capturing device 833, which will be described later, can also function as an input device by capturing the motion of the user's hand, user's finger, or the like. In this case, the pointing position may be determined depending on the motion of the hand or the direction of the finger.

The output device 817 includes a device capable of notifying visually or audibly the user of the acquired information. The output device 817 may be a display device such as a liquid crystal display (LCD), a plasma display panel (PDP), an organic electro-luminescence (EL) display, and a projector, a hologram display device, an audio output device such as a speaker and a headphone, as well as printer devices or the like. The output device 817 outputs the result obtained by the processing of the terminal 10 as a video such as a text or an image, or outputs it as audio such as a speech or sound. In addition, the output device 817 may include, in one example, a light for lighting up the surroundings.

The storage device 819 is a data storage device configured as an example of a storage portion of the terminal 10. The storage device 819 includes, in one example, a magnetic storage unit device such as hard disk drive (HDD), a semiconductor storage device, an optical storage device, and a magneto-optical storage device. The storage device 819 stores programs executed by the CPU 801, various data, various types of data obtained from the outside, and the like.

The drive 821 is a reader-writer for a removable recording medium 827 such as a magnetic disk, an optical disk, a magneto-optical disk, and a semiconductor memory, and is incorporated in the terminal 10 or externally attached thereto. The drive 821 reads the information recorded on the loaded removable recording medium 827 and outputs it to the RAM 805. In addition, the drive 821 writes a record in the loaded removable recording medium 827.

The connection port 823 is a port for directly connecting the device to the terminal 10. The connection port 823 may be, in one example, a universal serial bus (USB) port, an IEEE 1394 port, or a small computer device interface (SCSI) port. In addition, the connection port 823 may be, in one example, an RS-232C port, an optical audio terminal, or high-definition multimedia interface (HDMI, registered trademark) port. The connection of the external connection device 829 to the connection port 823 makes it possible to exchange various kinds of data between the terminal 10 and the external connection device 829.

The communication device 825 is, in one example, a communication interface including a communication device or the like, which is used to be connected to the communication network 931. The communication device 825 may be, in one example, a communication card for wired or wireless local area network (LAN), Bluetooth (registered trademark), or wireless USB (WUSB). In addition, the communication device 825 may be, in one example, a router for optical communication, a router for asymmetric digital subscriber line (ADSL), or a modem for various communications. The communication device 825 transmits and receives signals or the like using a predetermined protocol such as TCP/IP, in one example, with the Internet or other communication devices. In addition, the communication network 931 connected to the communication device 825 is a network connected by wire or wireless, and is, in one example, the Internet, home LAN, infrared communication, radio wave communication, satellite communication, or the like.

The image capturing device 833 is a device that captures a real space and generates a captured image, by using an image sensor such as charge-coupled device (CCD) or complementary-metal-oxide semiconductor (CMOS) and various members such as a lens for controlling imaging of a subject image on the image sensor. The image capturing device 833 can capture a still image or a moving image.

The sensor 835 is, in one example, various sensors such as an acceleration sensor, a gyro sensor, a geomagnetic sensor, an optical sensor, and a sound sensor. The sensor 835 acquires information related to the state of the terminal 10 such as the attitude of the casing of the terminal 10, and acquires information related to the surrounding environment of the terminal 10 such as brightness or noise around the terminal 10. The sensor 835 may also include a GPS sensor that receives global positioning system (GPS) signals and measures the latitude, longitude, and altitude of the device.

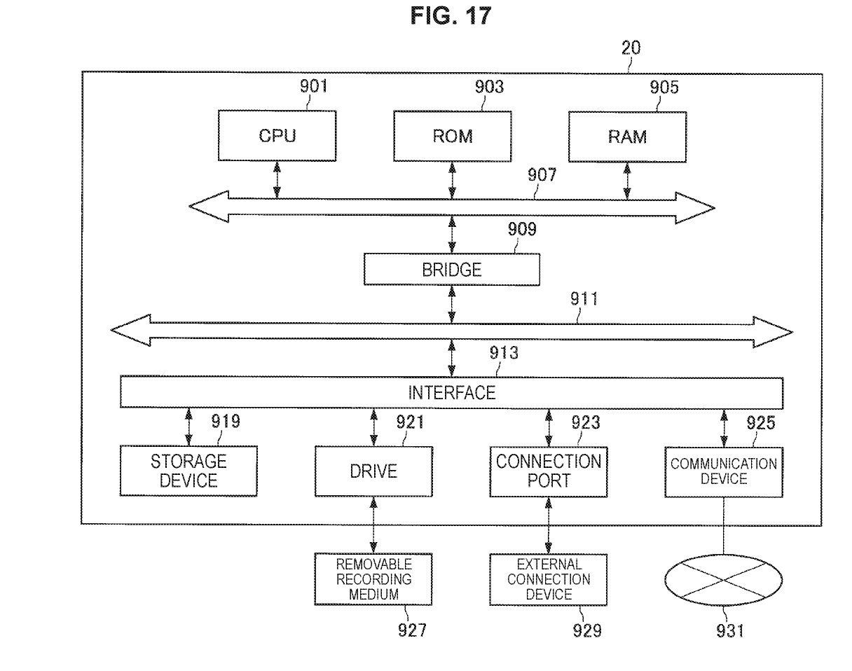

Next, the hardware configuration of the information processing device 20 according to an embodiment of the present disclosure is described with reference to FIG. 17. FIG. 17 is a block diagram illustrating a hardware configuration example of the information processing device 20 according to an embodiment of the present disclosure.

As illustrated in FIG. 17, the information processing device 20 includes a central processing unit (CPU) 901, a read-only memory (ROM) 903, and a random-access memory (RAM) 905. In addition, the information processing device 20 may include a host bus 907, a bridge 909, an external bus 911, an interface 913, a storage device 919, a drive 921, a connection port 923, and a communication device 925. In conjunction with, or in place of, the CPU 901, the information processing device 20 may have a processing circuit called a digital signal processor (DSP) or application specific integrated circuit (ASIC).

The CPU 901 functions as an arithmetic processing unit and a control unit, and controls the whole operation in the information processing device 20 or a part thereof in accordance with various programs recorded in the ROM 903, the RAM 905, the storage device 919, or a removable recording medium 927. The ROM 903 stores programs, operation parameters, or the like used by the CPU 901. The RAM 905 temporarily stores programs used in the execution by the CPU 901, parameters that vary as appropriate in the execution, or the like. The CPU 901, the ROM 903, and the RAM 905 are connected with each other via the host bus 907 that includes an internal bus such as a CPU bus. Furthermore, the host bus 907 is connected to the external bus 911 such as peripheral component interconnect/interface (PCI) bus via the bridge 909.

The storage device 919 is a data storage device configured as an example of a storage portion of the information processing device 20. The storage device 919 includes, in one example, a magnetic storage unit device such as hard disk drive (HDD), a semiconductor storage device, an optical storage device, and a magneto-optical storage device. The storage device 919 stores programs executed by the CPU 901, various data, various types of data obtained from the outside, and the like.

The drive 921 is a reader-writer for a removable recording medium 927 such as a magnetic disk, an optical disk, a magneto-optical disk, and a semiconductor memory, and is incorporated in the information processing device 20 or externally attached thereto. The drive 921 reads the information recorded on the loaded removable recording medium 927 and outputs it to the RAM 905. In addition, the drive 921 writes a record in the loaded removable recording medium 927.

The connection port 923 is a port for directly connecting the device to the information processing device 20. The connection port 923 may be, in one example, a universal serial bus (USB) port, an IEEE 1394 port, or a small computer system interface (SCSI) port. In addition, the connection port 923 may be, in one example, an RS-232C port, an optical audio terminal, or high-definition multimedia interface (HDMI, registered trademark) port. The connection of the external connection device 929 to the connection port 923 makes it possible to exchange various kinds of data between the information processing device 20 and the external connection device 929.

The communication device 925 is, in one example, a communication interface including a communication device or the like, which is used to be connected to a communication network 931. The communication device 925 may be, in one example, a communication card for wired or wireless local area network (LAN), Bluetooth (registered trademark), or wireless USB (WUSB). In addition, the communication device 925 may be, in one example, a router for optical communication, a router for asymmetric digital subscriber line (ADSL), or a modem for various communications. The communication device 925 transmits and receives signals or the like using a predetermined protocol such as TCP/IP, in one example, with the Internet or other communication devices. In addition, the communication network 931 connected to the communication device 925 is a network connected by wire or wireless, and is, in one example, the Internet, home LAN, infrared communication, radio wave communication, satellite communication, or the like.

### 2. CONCLUSION

As described above, according to the embodiment of the present disclosure, there can be provided the information processing device 20, which includes the acquisition unit 221 that acquires the input information B including at least the genetic test result 41B and the health examination result 42B and the computing unit 222 that acquires the weight of each of the plurality of comments on the basis of the category to which the input information B belongs and specifies the comment to be notified from the plurality of comments on the basis of the weight of each of the plurality of comments.

According to such a configuration, it becomes possible to generate the comment more suitable for the subject of the genetic test. Further, according to such a configuration, it becomes possible to prevent the variation of the comment caused by the difference in the medical doctors who generate the comment and reduce a burden of generating the comment by the medical doctor.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, a position of each component is not particularly limited as long as the operation of the information processing system 1 described above is achieved. For example, the example where the entire database 241 is stored in the information processing device 20 is described above. However, a part or all pieces of information in the database 241 may be stored in another information processing device (a second information processing device) different from the information processing device 20 (a first information processing device) and provided from such another information processing device to the information processing device 20.

Further, the example where all of the acquisition unit 221, the computing unit 222, and the notification unit 223 are included in the single information processing device 20 is described above. However, the acquisition unit 221, the computing unit 222, and the notification unit 223 may be distributed in a plurality of information processing devices.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An information processing device including:

an acquisition unit that acquires first input information that includes at least a first genetic test result and a first health examination result; and

a computing unit that acquires a weight of each of a plurality of data on a basis of a category to which the first input information belongs and specifies the data to be notified from the plurality of data on a basis of the weight of each of the plurality of data.

(2)

The information processing device according to (1), in which the computing unit updates the weight of the data to be notified by reflecting an improvement probability by the data to be notified on the weight of the data to be notified as feedback to notification of the data to be notified.

(3)

The information processing device according to (2), in which the computing unit calculates the improvement probability by the data to be notified on a basis of information indicating whether or not an improvement is made by the notification of the data to be notified.

(4)

The information processing device according to (3), in which the acquisition unit acquires the information indicating whether or not an improvement is made through a manual input.

(5)

The information processing device according to (3), in which the computing unit automatically calculates the information indicating whether or not an improvement is made.

(6)

The information processing device according to (5), in which the computing unit calculates the information indicating whether or not an improvement is made on a basis of the health examination result in a past and the health examination result at present.

(7)

The information processing device according to any one of (1) to (6), including:

a notification unit that notifies the data to be notified.

(8)

The information processing device according to any one of (1) to (7), in which the first input information further includes a first inquiry result.

(9)

The information processing device according to any one of (1) to (8), in which each of the plurality of data includes a comment provided from a medical doctor to a subject.

(10)

The information processing device according to any one of (1) to (9), in which the computing unit performs learning processing on a category to which an already stored second input information belongs and the data associated with the second input information and thereby calculates the weight of each of the plurality of data in each category.

(11)

The information processing device according to (10), in which the second input information includes at least a second genetic test result and a second health examination result.

(12)

The information processing device according to (11), in which the second input information further includes a second inquiry result.

(13)

The information processing device according to any one of (1) to (12), in which the computing unit specifies a corresponding weight on a basis of an occurrence probability of each of the plurality of data.

(14)

The information processing device according to any one of (1) to (13), in which the computing unit specifies the data having a largest weight among the weights of the plurality of data as the data to be notified.

(15)

The information processing device according to (1), in which the computing unit adds new data to the plurality of data in a case where the new data is added to a report describing the data to be notified.

(16)

The information processing device according to any one of (1) to (15), in which the computing unit updates an occurrence probability of each of the plurality of data in a case where the data to be notified is notified.

(17)

An information processing method including:
acquiring first input information that includes at least a first genetic test result and a first health examination result; and acquiring a weight of each of a plurality of data on a basis of a category to which the first input information belongs and specifying the data to be notified from the plurality of data on a basis of the weight of each of the plurality of data by a processor.
   (18)

A program that causes a computer to function as an information processing device that includes

an acquisition unit that acquires first input information that includes at least a first genetic test result and a first health examination result, and

a computing unit that acquires a weight of each of a plurality of data on a basis of a category to which the first input information belongs and specifies the data to be notified from the plurality of data on a basis of the weight of each of the plurality of data.

### REFERENCE SIGNS LIST

1 information processing system
10 terminal
20 information processing device
31A, 31B comment
41A, 41B genetic test result
42A, 42B health examination result
43A, 43B inquiry result
110 operation unit
120 control unit
130 communication unit
140 storage unit
150 display unit
220 control unit
221 acquisition unit
222 computing unit
223 notification unit
230 communication unit
240 storage unit
241 database

1. An information processing device comprising:
an acquisition unit that acquires first input information that includes at least a first genetic test result and a first health examination result; and
a computing unit that acquires a weight of each of a plurality of data on a basis of a category to which the first input information belongs and specifies the data to be notified from the plurality of data on a basis of the weight of each of the plurality of data.

2. The information processing device according to claim 1, wherein the computing unit updates the weight of the data to be notified by reflecting an improvement probability by the data to be notified on the weight of the data to be notified as feedback to notification of the data to be notified.

3. The information processing device according to claim 2, wherein the computing unit calculates the improvement probability by the data to be notified on a basis of information indicating whether or not an improvement is made by the notification of the data to be notified.

4. The information processing device according to claim 3, wherein the acquisition unit acquires the information indicating whether or not an improvement is made through a manual input.

5. The information processing device according to claim 3, wherein the computing unit automatically calculates the information indicating whether or not an improvement is made.

6. The information processing device according to claim 5, wherein the computing unit calculates the information indicating whether or not an improvement is made on a basis of the health examination result in a past and the health examination result at present.

7. The information processing device according to claim 1, comprising:
a notification unit that notifies the data to be notified.

8. The information processing device according to claim 1, wherein the first input information further includes a first inquiry result.

9. The information processing device according to claim 1, wherein each of the plurality of data includes a comment provided from a medical doctor to a subject.

10. The information processing device according to claim 1, wherein the computing unit performs learning processing on a category to which an already stored second input information belongs and the data associated with the second input information and thereby calculates the weight of each of the plurality of data in each category.

11. The information processing device according to claim 10, wherein the second input information includes at least a second genetic test result and a second health examination result.

12. The information processing device according to claim 11, wherein the second input information further includes a second inquiry result.

13. The information processing device according to claim 1, wherein the computing unit specifies a corresponding weight on a basis of an occurrence probability of each of the plurality of data.

14. The information processing device according to claim 1, wherein the computing unit specifies the data having a largest weight among the weights of the plurality of data as the data to be notified.

15. The information processing device according to claim 1, wherein the computing unit adds new data to the plurality of data in a case where the new data is added to a report describing the data to be notified.

16. The information processing device according to claim 1, wherein the computing unit updates an occurrence probability of each of the plurality of data in a case where the data to be notified is notified.

17. An information processing method comprising:
acquiring first input information that includes at least a first genetic test result and a first health examination result; and
acquiring a weight of each of a plurality of data on a basis of a category to which the first input information belongs and specifying the data to be notified from the plurality of data on a basis of the weight of each of the plurality of data by a processor.

18. A program that causes a computer to function as an information processing device that includes
an acquisition unit that acquires first input information that includes at least a first genetic test result and a first health examination result, and
a computing unit that acquires a weight of each of a plurality of data on a basis of a category to which the first input information belongs and specifies the data to be notified from the plurality of data on a basis of the weight of each of the plurality of data.
